(19) United States
(12) Patent Application Publication
(10) Pub. No.: US 2009/0239288 A1
(43) Pub. Date: Sep. 24, 2009

(54) INTEGRATED MEMBRANE SEPARATION - BIOREACTOR FOR SELECTIVE REMOVAL OF ORGANIC PRODUCTS AND BY-PRODUCTS

(75) Inventors: Shiguang Li, Mount Prospect, IL (US); Qinbai Fan, Chicago, IL (US); William E. Liss, Libertyville, IL (US); John J. Lewnard, Westford, MA (US)

Correspondence Address: MARK E. FEJER;GAS TECHNOLOGY INSTITUTE, 1700 SOUTH MOUNT PROSPECT ROAD, DES PLAINES, IL, 60018, US

(73) Assignee: GAS TECHNOLOGY INSTITUTE, Des Plaines, IL (US)

(21) Appl. No.: 12/134,315

(22) Filed: Jun. 6, 2008

Related U.S. Application Data
(63) Continuation-in-part of application No. 12/051,207, filed on Mar. 19, 2008.

(51) Int. Cl. A62D 3/02; C12M 1/00

(52) U.S. Cl. 435/262; 435/297.1

(57) ABSTRACT

A system for selective removal of metabolites from an aqueous broth including a bioreactor vessel containing at least one live culture producing at least one metabolite disposed within an aqueous broth, and a membrane module having a mixed matrix membrane suitable for selective removal of the at least one metabolite from the aqueous broth. The membrane is made of mixtures of hydrophobic zeolite and carbon on a porous substrate. In accordance with one particularly preferred embodiment of this invention, the membrane module is integral with the bioreactor vessel.

### BACKGROUND OF THE INVENTION

1. Field of the Invention

This invention relates in one aspect to a method and system for producing metabolites. In another aspect, this invention relates to a system for producing metabolites comprising a bioreactor and a separation membrane integral with the bioreactor. In another aspect, this invention relates to a method for the selective removal of metabolites from an aqueous broth within a bioreactor producing the metabolites.

2. Description of Related Art

A bioreactor is a vessel containing live microbial cultures of yeast, bacteria, or similar cells which produce metabolites such as ethanol, butanol, acetic acid, citric acid and the like. The metabolites are typically water-soluble, have vapor pressures similar to water, and may form azeotropes with water. In some cases, these metabolites are the primary products of the bioreaction process. For example, biomass-to-ethanol conversion processes involve the fermentative production of ethanol from sugars comprising the biomass. Depending upon the biomass source and hydrolysis process, the concentration of ethanol in the fermentation (aqueous) broth ranges from about 3 to about 15 wt %. Thus, in order to produce fuel grade ethanol, the ethanol in the fermentation broth must be removed from the broth. In other cases, the metabolites may be co-products or even undesirable by-products. However, in all cases, the accumulation of metabolites within the aqueous broth disposed within the bioreactor vessel will ultimately inhibit the biological activity of the cultures, resulting in a reduction in the production of the desired product and/or co-product metabolites. Thus, it is necessary to remove the metabolites from the aqueous broth, preferably without disruption of the biologically active cells in the bioreactor vessel, to maintain high productivity and/or yields of metabolites.

Conventional solutions to address this problem include decoupling of the bioreactor and separation operations, and the use of energy intensive separation steps. For example, many conventional systems operate with sequential placement of the bioreactor and downstream product separation steps. The operations are distinct, and typically, effluents from the separation are discarded rather than returned to the bioreactor. For example, in the batch fermentation of corn to produce ethanol, the entire batch is dumped once the ethanol concentration has reached about 13 wt %, at which point the yeast used in the fermentation process is no longer active. The reactor effluent is distilled to produce about 95 wt %, and the still bottoms are rejected as brewer's yeast. Some systems, such as the BioStil process, operate with a bleed stream from the bioreactor that passes through a centrifuge to recover the active biomass. The recovered active biomass is then returned to the bioreactor, with the centrate being sent to the distillation column. Some fraction of the aqueous-rich stream, referred to as weak beer, is returned to the bioreactor, with part rejected as stillage. Heat exchangers are required to reduce the weak beer temperature to a suitable range prior to recycle to the bioreactor. In both of these examples, flash (single stage) or distillation (multi-stage) operations are used for recovering dilute ethanol from the fermentation broth. The ethanol may be further concentrated using molecular sieve adsorption to remove water down to fuel grade levels. The primary disadvantages with all of these systems include high energy consumption and excessive amount of wastewater discharged from the distillation columns.

An alternative integrated process includes vacuum fermentation, which is most effective for removing compounds that have vapor pressures substantially higher than water. However, these systems typically involve high operating costs to maintain the required vacuum and may limit the options for biological species due to the temperature requirements. The process may also result in the accumulation of heavier compounds in the broth.

A non-thermal, integrated process is in-situ solvent extraction in which a solvent having high affinity for the target organic compound is fed to the bioreactor and continuously removed. Ideally, the solvent forms an immiscible phase or can be selectively removed by a membrane. However, these systems have found limited applicability because the solvents must have a high affinity for the target organic compound and must not be toxic to the system biology.

### SUMMARY OF THE INVENTION

Accordingly, it is one object of this invention to provide a method and system for removal of metabolites from a bioreactor which method and system operate at conditions that do not adversely affect the biology, which may be sensitive to temperature, shear, pH and the like, of the metabolite formation process.

It is another object of this invention to provide a method and system for removal of metabolites from a bioreactor which requires relatively low energy input compared with conventional methods and systems, for example, the selective removal of higher boiling compounds in the fermentation broth without needing to boil off the water.

It is yet a further object of this invention to provide a method and system for removal of metabolites form a bioreactor which integrates the separation system with the bioreactor to reduce capital and operating costs. Integration of the separation system with the bioreactor permits batch, semi-batch, and continuous operations, such as perfusion systems, to operate at higher yields or conversions by maintaining lower net concentrations of inhibitory metabolites throughout the metabolite production process.

These and other objects of this invention are addressed by a system for selective removal of metabolites from an aqueous broth comprising a bioreactor vessel containing at least one live culture producing at least one metabolite disposed within an aqueous broth, and a membrane module comprising a mixed matrix membrane suitable for selective removal of the at least one metabolite from the aqueous broth having a mixed matrix membrane feed side in contact with the aqueous broth and having a mixed matrix membrane permeate side. The membrane comprises mixtures of hydrophobic zeolite and/or carbon on a porous substrate. In accordance with one particularly preferred embodiment of this invention, the membrane module is integral with the bioreactor vessel.

In the operation of the system, the mixed matrix membrane feed side of a mixed matrix membrane integral with a bioreactor vessel is contacted with a fermentation or aqueous broth comprising at least one metabolite. The at least one metabolite is selectively passed through the mixed matrix membrane to the mixed matrix membrane permeate side of the mixed matrix membrane, producing a concentrated said metabolite separate from the broth.

### BRIEF DESCRIPTION OF THE DRAWINGS

These and other objects and features of this invention will be better understood from the following detailed description taken in conjunction with the drawings wherein:

FIG. 1 is a schematic diagram of a system for selective removal of metabolites from an aqueous broth within a bioreactor producing the metabolites in accordance with one embodiment of this invention; and

FIG. 2 is a cross-sectional view of a portion of a bioreactor vessel wall with an integrated membrane module for separation of metabolites in accordance with one embodiment of this invention.

### DETAILED DESCRIPTION OF THE PRESENTLY PREFERRED EMBODIMENTS

As used herein, the term “bioreactor” or “bioreactor vessel” refers to any apparatus, such as a large fermentation chamber, for growing organisms such as bacteria or yeast under controlled conditions for production of substances such as pharmaceuticals, antibodies, or vaccines, or for the bioconversion of organic waste.

As used herein, the term “metabolite” refers to an organic compound or molecule that is a starting material in, an intermediate in, or an end product of, metabolism.

The invention disclosed herein involves the integration of a bioreactor with a membrane which selectively removes organic molecules from an aqueous media or broth within the bioreactor. The bioreactor contains live cultures of yeast, bacteria or similar microbial cultures which, depending upon conditions within the bioreactor, produce a plethora of metabolites which may be target products, e.g. ethanol co-products, e.g. butanol, and undesirable metabolism inhibitory by-products, e.g. acetaldehyde, glycerol, formic acid, lactic acid, acetic acid, 1-propanol, 2-methyl-1-butanol, and 2,3-butanediol to name a few. The metabolites may be polar, non-polar, volatile and/or non-volatile compounds. And, depending upon the mix of target products, co-products and inhibitory by-products, membrane separation processes employed in accordance with various embodiments of this invention may include pervaporation, which is described in our co-pending U.S. patent application Ser. No. 12/051,207 and which is incorporated by reference in its entirety herein, perfusion, e.g. reverse osmosis, and perstraction, the use of a sweep fluid on the permeate side of the membrane.

The membrane for selective removal of organic compounds from the aqueous media or broth within the bioreactor vessel in accordance with one embodiment of this invention is a mixed matrix membrane comprising at least one layer of a porous carbon-based structure and a plurality of silicalite crystals dispersed on the surface of the porous carbon-based structure and/or within the interior of the porous carbon-based structure. In accordance with one embodiment of this invention, the silicalite crystals form a substantially continuous layer on the surface of the carbon-based structure. In accordance with one preferred embodiment of this invention, the substantially continuous layer of silicalite crystals is a monolayer, i.e. a layer having a thickness of one molecule. Requirements for the porous carbon-based structure are thermal stability and stiffness. Thermal stability is important due to the high temperatures, e.g. 370° C., required to produce the membrane. The porous carbon-based structure also should be substantially inflexible because the continuous zeolite layer applied to the porous carbon-based structure is brittle. In accordance with one embodiment of this invention, the porous carbon-based structure is a structure selected from the group consisting of a composite porous graphite foam, carbon fiber paper, carbon cloth, and combinations thereof.

Porosity of the carbon-based structure is in the range of about 15% to about 80%. It will, however, be appreciated that as the porosity of the carbon-based structure decreases the transport resistance of the membrane will increase; thus, porosities towards the higher end of the range, about 30% to about 80%, are generally preferred. Porosities above about 80% are undesirable due to the potential for allowing compounds other than ethanol to penetrate through the membrane. Pore sizes of the pores of the carbon-based structure is also a critical factor; pore sizes that are too large increase the potential for the water in the ethanol/water mixture to pass through the membrane whereas pore sizes that are too small will prevent the ethanol from passing through the membrane. Accordingly, in accordance with one embodiment of this invention, the pore sizes of the pores of the porous carbon-based structure are in the range of about 0.1 μm to about 5.0 μm. In accordance with one preferred embodiment, pore sizes are in the range of about 0.2 μm to about 0.5 μm.

Production of thin, defect-free membranes in accordance with this invention requires the use of nano-scale silicalite seeds or particles as a starting material. In accordance with one embodiment of this invention, particle sizes of the seed silicalite particles is in the range of about 50 nm to about 500 nm. In accordance with one preferred embodiment, particle sizes of the seed silicalite particles are in the range of about 80 nm to about 120 nm.

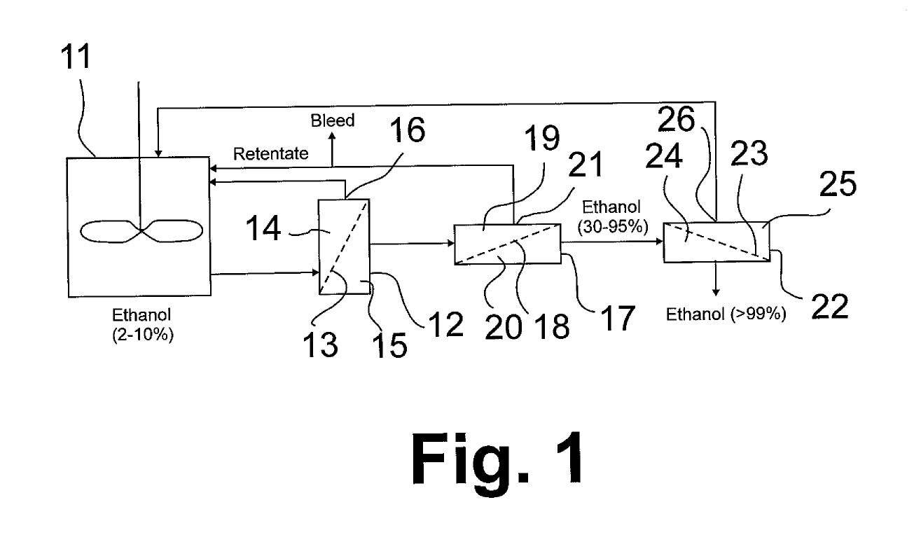

FIG. 1 shows a schematic diagram of a process and system for recovering ethanol during fermentation in a bioreactor vessel 11 to maintain ethanol concentrations in the bioreactor vessel at lower levels (in the range of about 2-10 wt %), which promotes more efficient production of ethanol by the microbes employed in the fermentation process while increasing the product ethanol concentration to greater than 99 wt %. As shown therein, the system comprises a microfiltration unit 12 comprising a microfiltration membrane 13 having a microfiltration membrane feed side 14 and a microfiltration membrane permeate side 15, which microfiltration unit removes solids in the process stream to prevent fouling of downstream membrane units 17 and 22. Suitable microfiltration membranes have pore sizes in the range of about 0.1 to about 10 microns and may be made of polymers, e.g. polysulfone and polyvinylidine fluoride, and inorganic materials, e.g. Al₂O₃. Microfiltration unit 12 further comprises microfiltration unit retentate outlet 16 in fluid communication with bioreactor vessel 11 by way of which fluid communication retentate in microfiltration unit 12 is returned to bioreactor vessel 11. Disposed downstream of microfiltration unit 12 is hydrophobic membrane unit 17 comprising hydrophobic mixed matrix membrane 18 having mixed matrix membrane feed side 19, which is in fluid communication with microfiltration permeate side 15 of microfiltration membrane 13, and mixed matrix membrane permeate side 20. Hydrophobic membrane unit 17 further comprises hydrophobic membrane unit retentate outlet 21 in fluid communication with bioreactor vessel 11 by way of which fluid communication retentate in hydrophobic membrane unit 17 is returned to bioreactor vessel 11. Disposed downstream of hydrophobic membrane unit 17 is hydrophilic membrane unit 22 comprising hydrophilic membrane 23 having a hydrophilic membrane feed side 24 in fluid communication with mixed matrix membrane permeate side 20 of mixed matrix membrane 18 and hydrophilic membrane permeate side 25. Hydrophilic membrane 23 in accordance with one embodiment of this invention is a commercially available NaA hydrophilic membrane. Hydrophilic membrane unit 22 further comprises hydrophilic membrane unit permeate outlet 26 in fluid communication with bioreactor vessel 11 by way of which fluid communication permeate from said hydrophilic membrane permeate side 25 of hydrophilic membrane 23 is returned to bioreactor vessel 11.

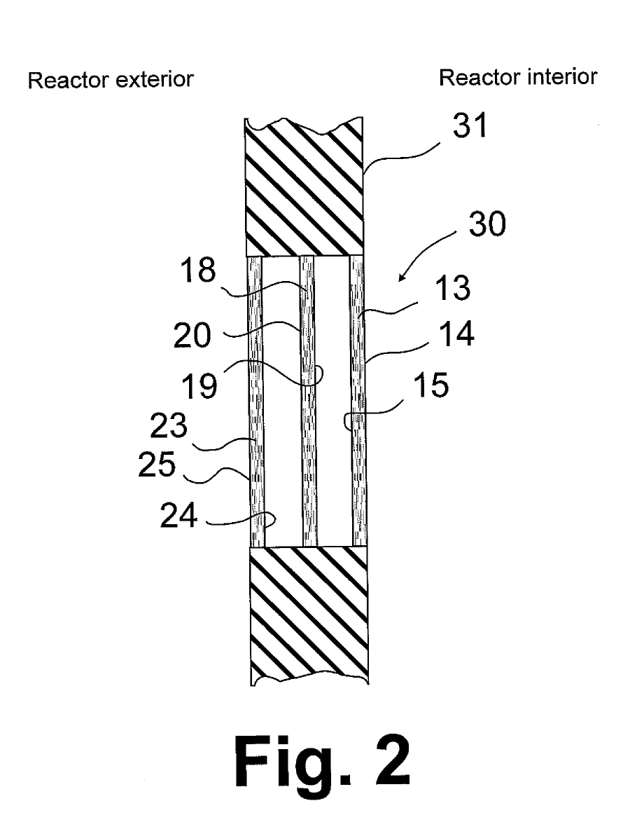

In accordance with one embodiment of this invention, mixed matrix membrane 18 is disposed within a membrane module 30 which is integral with wall 31 of bioreactor vessel 11 as shown in FIG. 2. In accordance with one embodiment of this invention, at least one of microfiltration membrane 13 and hydrophilic membrane 23 is disposed within membrane module 30 together with mixed matrix membrane 18. Integration of the separation operations of the membranes with the bioreactor vessel in this manner provides several benefits. One primary benefit is a greatly reduced fermentor volumetric productivity which translates to lower installed fermentor capacity or greater output for a given fermentor volume. In addition, as previously indicated, integration of the membrane modules with the bioreactor allows batch, semi-batch, and continuous operations, such as perfusion systems, to operate at higher yields or conversions by the continuous removal of inhibitory products within the bioreactor while maintaining the active biological processes within the bioreactor. A further benefit of this arrangement is the fact that the hydrophobic membrane does not require thermal energy to accomplish the separation. For ethanol production, it is expected that the method and integrated system of this invention will result in about a 40% reduction in energy consumption compared with current conventional distillation processes. Yet a further benefit of the system of this invention results from the fact that each of the membranes employed comprises inorganic materials, thereby providing thermal and chemical stability in the fermentation broth, which translates into long service lives. In addition, due to the high thermal and chemical stability of the membranes, they can easily be sterilized to ensure system maintenance. The use of perfusion membranes, which is like a pump-around loop, can facilitate flushing options to minimize fouling. For example, the membrane may be isolated from the bioreactor, flushed backwards to drain, and then restored to the reactor operation. The pump-around loop may also be used to sterilize the fermentation broth and remove other materials from the system, i.e. dead cells if the yeast/bacteria are immobilized and other chemical species that inhibit fermentation, and sterilize the fermentation broth.

It will be appreciated by those skilled in the art that separation membranes such as those used in the method and system of this invention require a driving force to drive the target metabolites through the membranes. In accordance with one embodiment of this invention, a pervaporation mode, where partial fugacity is used to drive volatile target metabolites through the membranes, may be used. The lower fugacity on the permeate side may be achieved by condensing vapor with a cold trap.

In accordance with one embodiment of this invention, the feed side of the membrane is pressurized so as to create a reverse osmosis mode.

In accordance with yet another embodiment of this invention, perstraction, where a sweep fluid is employed on the permeate side of the membrane, is used. The particular sweep fluid employed will vary depending upon the particulars of the separation process. For example, the sweep fluid typically will have properties to facilitate separation of the desired species from the sweep fluid such as forming a 2-phase fluid (ethanol/PEG and other liquids which also have low permeability in the membrane and low toxicity to the biology). Alternatively, the sweep fluid may have very different volatility/boiling points (i.e. ethanol/octanol). Again, it is important that the sweep fluid have low permeability and low toxicity. Alternatively, the sweep fluid may have a chemical interaction to drive the effective concentration on the permeate side to zero. A good example is the use of (basic) amines with membrane contactors for CO₂ removal. In accordance with one embodiment which is particularly useful for non-volatile compounds, the sweep fluid comprises one or more adsorbents, e.g. polar adsorbents such as zeolites or non-polar adsorbents such as carbon, suspended in an isotonic fluid (essentially the bioreactor media without the biology). In this case, the biology is not affected by the reverse flux of the sweep fluid.

Although ethanol is the primary product of fermentation, numerous co-products also inhibit biological action and in some cases are more difficult to remove than ethanol. By-products may be volatile like alcohols and ketones or non-volatile like acids. The main products which are not volatile include acids, in particular citric and lactic acid, which are produced on a very large scale and use precipitation for recovery. Removal of citric acid may be accomplished in accordance with one embodiment of this invention using a sweep fluid comprising an isotonic fluid containing basic zeolite. The isotonic fluid uses media components to ensure no osmotic pressure differentials and minimizes flux of water and ions across the membrane. The zeolite forms a weak chemical bond with the citric acid and is regenerated with stronger acid and then treated with a base, such as ion exchange resins, to recover the acid. Ideally, stronger acids and bases will have counter-ions that are used in the isotonic solution, i.e. NaOH and HCl.

### Example

In this example, silicalite/carbon membranes were prepared on porous stainless steel supports. The porous stainless steel tubular supports having a pore size of about 0.8 microns were purchased from Pall Corporation. Nonporous, stainless steel tubes were welded onto each end of the supports. The permeate area was approximately 7.8 cm². Before synthesis, the supports were boiled in de-ionized water for 1 hour and dried at 373° K. for 1 hour. The stainless steel supports were then seeded by rubbing the inside surface with nano-scale silicalite crystals. The seeded tubes were placed in an autoclave and filled with synthesis gel having a molar composition of 1.0 TPAOH (tetrapropylammonium hydroxide): 19.5 SiO₂: 438 H₂O, for hydrothermal treatment. The synthesis gel was prepared by adding colloidal silica sol (Ludox AS40, 40% aqueous solution) to H₂O at room temperature for 5 minutes. Thereafter, the template, TPAOH, was added to the mixture. The solution was sealed, stirred, and aged for approximately 3 hours at room temperature before use.

The hydrothermal synthesis was carried at 468° K. for a certain time shown in Table 1. After synthesis, the membranes were washed with distilled water at room and dried at 373° K. in an oven for 1 hour. The synthesis was repeated until an uncalcined membrane, after drying at 373° K., was impermeable to N₂ for a 138-kPa pressure drop at room temperature. Because the TPAOH template filled the silicalite pores during synthesis and thus blocked light gas permeation, a membrane with no defects should be impermeable. However, the template could also fill pores that are larger than the silicalite pores.

After the zeolite synthesis was completed, the membranes were washed, dried, and calcined in air to remove the template from the pores. The calcination procedure was carried out in a muffle furnace resulting in the formation of a silicalite/carbon composite membrane. The calcination conditions are shown in Table 1. Note that the calcination temperatures were lower than those (typically 550° C.) reported in literature.

TABLE 1
Crystallization conditions for silicalite/carbon on stainless steel membranes

| | Crystallization | |
|---|---|---|
| Membrane | Conditions | Calcination Conditions |
| M4 | Two synthesis steps: | Heating and cooling rates of |
| | | 0.6° K/min. |
| | First step-24 hrs | Maximum temperature-673° K; |
| | Second step-24 hrs. | Membrane held at temperature for |
| | | 10 hrs. |
| M5 | Two synthesis steps: | Heating and cooling rates of |
| | | 0.6° K/min. |
| | First step-6 hrs | Maximum temperature-673° K; |
| | Second step-6 hrs. | Membrane held at temperature for |
| | | 50 hrs. |

### Example

In this example, silicalite/carbon composite membranes were used to separate 3 wt. % ethanol from water by pervaporation. The membranes were sealed in a permeation cell with Viton O-rings. The ethanol/water liquid was fed to one side of the membrane by a pump. A vacuum pump evacuated the permeate side of the membrane to a pressure of approximately 0.2 kPa, and the pump was then valved off during pervaporation measurements. A liquid nitrogen cold trap condensed the permeate vapor and maintained the vacuum on the permeate side. A permeate sample was usually collected and weighted every hour to determine the flux. Permeate concentrations were measured by off-line HPLC. The separation properties of the silicalite/carbon composite membranes are shown in Table 2. As can be seen, membranes M4 and M5 were selective for ethanol over water since the ethanol concentrations were higher in the permeate than in the feed concentration of 3 wt. %.

TABLE 2
Separation properties of the silicalite/carbon membrane

| | Separation of 3 wt % ethanol/water | |
|---|---|---|
| | | Ethanol permeate |
| Membrane | Flux (kg/m²/h) | concentration (wt %) |
| M4 | 0.16 | 30 |
| M5 | 0.36 | 38 |

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

We claim:

1. A system for selective removal of metabolites from an aqueous broth comprising:
a bioreactor vessel containing at least one live culture producing at least one metabolite disposed within an aqueous broth; and
a membrane module comprising a mixed matrix membrane suitable for selective removal of said at least one metabolite from said aqueous broth having a mixed matrix membrane feed side in contact with said aqueous broth and having a mixed matrix membrane permeate side.

2. The system of claim 1, wherein said membrane module is integral with said bioreactor vessel.

3. The system of claim 1, wherein said membrane module comprises a microfiltration membrane disposed upstream of said mixed matrix membrane having a microfiltration membrane feed side in contact with said aqueous broth and having a microfiltration membrane permeate side in fluid communication with said mixed matrix membrane feed side.

4. The system of claim 1, wherein said membrane module comprises an hydrophilic membrane disposed downstream of said mixed matrix membrane having an hydrophilic membrane fuel side in fluid communication with said mixed matrix membrane permeate side and having an hydrophilic membrane permeate side.

5. The system of claim 3, wherein said membrane module comprises return means for returning retentate from said microfiltration membrane feed side to said aqueous broth in said bioreactor vessel.

6. The system of claim 1, wherein said membrane module comprises return means for returning retentate from said mixed matrix membrane feed side to said aqueous broth in said bioreactor vessel.

7. The system of claim 4, wherein said membrane module comprises return means for returning permeate from said hydrophilic membrane permeate side to said aqueous broth in said bioreactor vessel.

8. The system of claim 1, wherein said mixed matrix membrane comprises at least one hydrophobic zeolite and carbon on a porous substrate.

9. The system of claim 1 further comprising pressurizing means for pressurizing said mixed matrix membrane feed side of said mixed matrix membrane.

10. The system of claim 1 further comprising sweep means for providing a sweep fluid to said mixed matrix membrane permeate side of said mixed matrix membrane.

11. The system of claim 8, wherein said porous substrate comprises one of a porous carbon-based structure and a porous stainless steel structure.

12. A method for in-situ removal of metabolites from a mixture comprising said metabolites disposed within a bioreactor vessel, the method comprising the steps of:
contacting a mixed matrix membrane feed side of a mixed matrix membrane integral with said bioreactor vessel with said mixture; and
passing substantially only at least one of said metabolites through said mixed matrix membrane to a mixed matrix membrane permeate side of said mixed matrix membrane, producing a concentrated said at least one metabolite on said mixed matrix membrane permeate side.

13. The method of claim 12, wherein said at least one metabolite is removed from said mixture at a rate one of greater than and equal to a production rate of said at least one metabolite in said bioreactor vessel.

14. The method of claim 13, wherein said at least one metabolite is selected from the group consisting of primary metabolic products, metabolic co-products, metabolic by-products, and mixtures thereof produced in said bioreactor vessel.

15. The method of claim 14, wherein said metabolites are volatile compounds.

16. The method of claim 14, wherein said metabolites are non-volatile compounds.

17. The method of claim 16, wherein said metabolites are polar, non-volatile compounds.

18. The method of claim 12, wherein retentate on said mixed matrix membrane feed side of said mixed matrix membrane is returned to said mixture in said bioreactor vessel.

19. The method of claim 12, wherein said mixture contacts a microfiltration membrane feed side of a microfiltration membrane disposed upstream of said mixed matrix membrane, resulting in removal of components capable of fouling said mixed matrix membrane from said mixture.

20. The method of claim 12, wherein said mixed matrix membrane permeate side of said mixed matrix membrane is at a lower pressure than said mixed matrix membrane feed side of said mixed matrix membrane.
